(19) United States
(12) Patent Application Publication
(10) Pub. No.: US 2011/0208223 A1
(43) Pub. Date: Aug. 25, 2011

(54) SURGICAL INSTRUMENT AND METHOD OF USE FOR PERFORMING TISSUE RESECTION

(76) Inventor: Stephen SOLOMON, New York, NY (US)

(21) Appl. No.: 13/005,093

(22) Filed: Jan. 12, 2011

Related U.S. Application Data
(60) Provisional application No. 61/294,319, filed on Jan. 12, 2010.

(51) Int. Cl. A61B 17/32

(52) U.S. Cl. 606/167

(57) ABSTRACT

A surgical instrument is disclosed. The surgical instrument includes a first cutting element, a second cutting element, a guide member and an actuation mechanism with the guide member being attached to either the first cutting element, second cutting element or the actuation mechanism. The first and second cutting elements are operatively connected to the actuation mechanism with the actuation mechanism adapted to move at least one of the two cutting elements to resection tissue. The actuation mechanism includes a handle, trigger grip and at least one connecting member that connects the trigger grip to either one or both of the cutting elements to cause movement of the cutting elements when the trigger grip is pressed. A surgical method for performing an image-guided resection of diseased tissue is also disclosed. Further, a kit for tissue resection is disclosed that includes a plurality of surgical instruments and various configured elongated pins.

### CROSS-REFERENCE TO RELATED APPLICATIONS

This application claims priority from pending U.S. Provisional Patent Application 61/294,319 filed on Jan. 12, 2010 [Attorney Ref. 3314.003P], the disclosure of which is included by reference herein in its entirety.

### TECHNICAL FIELD

The present invention relates generally to surgical instrumentation, and more specifically, but not exclusively, to general surgery and cardio-thoracic resection instrumentation and techniques used for the targeting and removal of diseased tissue.

### BACKGROUND OF THE INVENTION

Historically, surgical resection of diseased tissue has been performed using anatomic resection techniques. Problems associated with these techniques include excess removal of healthy, viable tissue that could lead to unnecessary loss of function and compromised results.

The goals of limited or non-anatomic surgical techniques is to preserve as much surrounding healthy tissue as possible while removing the targeted diseased areas. Many challenges existing with the non-anatomic surgical technique, these include, but are not limited to: inadvertent diseased tissue remaining post-resection and failure to avoid vascular structures coursing though the target tissue area.

A need remains for the development of a guided surgical instrument to be used to facilitate the performance of the non-anatomic surgical procedure and further refinement of the corresponding non-anatomic surgical procedure, including the use of image guidance systems.

### SUMMARY OF THE INVENTION

Advancement of the state of the surgical instrumentation that are to be used to perform image-guided surgical resections and associated surgical techniques is believed desirable. The embodiment of the invention described herein satisfies the need for improvements to surgical instruments used to perform limited or non-anatomic surgical resections, like for example, the removal of a cancerous tumor within a lobe of a lung.

The present invention provides in one aspect, a surgical instrument for performing an image-guided, non-anatomic resection that includes a first cutting element and a second cutting element, a guide member and an actuation mechanism. The guide member is attached to either the first cutting element, the second cutting element or the actuation mechanism. In one aspect of the invention, the first cutting element and the second cutting element are operatively connected to the actuation mechanism, the actuation mechanism adapted to move either the first cutting element and the second cutting together or separately to facilitate the resection of tissue.

The present invention provides in yet another aspect, a surgical method for performing an image-guided, non-anatomic resection of diseased tissue. The method may include the step of creating at least one opening in the skin of a patient. The method may also have the step of obtaining a guided surgical instrument that has a first cutting element and a second cutting element, a guide member and an actuation mechanism. The guide member is attached to either the first cutting element, the second cutting element or the actuation mechanism. In one aspect of the invention, the first cutting element and the second cutting element are operatively connected to the actuation mechanism, the actuation mechanism adapted to move either the first cutting element and the second cutting together or separately to facilitate the resection of tissue. The method may further include the step of obtaining an image guidance system. The method may also include the step of inserting a plurality of needles into the patient through the at least one opening using the image guidance system to direct placement of the needles. The needles may include a first needle being inserted into the diseased tissue of the patient and a second needle and a third needle being inserted into the patient, the second and third needle being configured to receive the guide member of the surgical instrument and are oriented in a manner to define a cutting axis to be followed by the first and second cutting elements of the surgical instrument. The method may further have the step of moving the guide member relative to the second or third needle allowing the operator to move at least one of the first and second cutting elements of the surgical instrument to cut tissue.

Yet a further aspect of the present invention provides a tissue resection kit. The kit may include a plurality of surgical instruments for use in resecting targeted diseased tissue, each of the plurality of surgical instruments include two cutting elements, a guide member and an actuation mechanism. The guide member is attached to either of the two cutting elements or the actuation mechanism. In one aspect of the invention, the two cutting elements are operatively connected to the actuation mechanism with the actuation mechanism adapted to move either of the cutting elements together or separately to assist with resecting tissue. The kit further includes a plurality of various sized elongated needles.

Further, additional features and advantages are realized through the techniques of the present invention. Other embodiments and aspects of the invention are described in detail herein and are considered a part of the claimed invention.

### BRIEF DESCRIPTION OF THE DRAWINGS

The subject matter which is regarded as the invention is particularly pointed out and distinctly claimed in the claims at the conclusion of the specification. The foregoing and other objects, features and advantages of the invention are apparent from the following detailed description taken in conjunction with the accompanying drawings in which:

FIG. 1 is a side, elevational view of one embodiment of a surgical instrument; in accordance with an aspect of the present invention;

FIG. 2 is an elevational view from the proximal end of the surgical instrument of FIG. 1, in accordance with an aspect of the present invention;

FIG. 3 is an anterior view of the two guide needles in position along the margins of the diseased tissue in a lung and defining a wedge shape for establishing the cutting axis and the central traction needle positioned to hold and capture the targeted diseased tissue for resection, in accordance with an aspect of the present invention;

FIG. 4 is an anterior view of the two guide needles in position defining a wedge shape around the diseased tissue with the surgical instrument of FIG. 1 positioned along one of the guide pins and the central traction needle positioned to hold and capture the targeted diseased tissue for resection, in accordance with an aspect of the present invention;

FIG. 5 is a chart outlining the steps of the surgical method of performing an image guided non-anatomic resection of diseased tissue, in accordance with an aspect of the present invention;

FIG. 6 is a photograph of a side, elevational view of the surgical instrument of FIG. 1, in accordance with an aspect of the present invention;

FIG. 7 is a photograph of an enlarged, side, elevational view of the proximal end of the surgical instrument of FIG. 1 showing the guide member and the two cutting elements, in accordance with an aspect of the present invention;

FIG. 8 is a photograph of an enlarged, distal perspective view of the surgical instrument of FIG. 1, showing the handle and trigger grip of the actuation mechanism, in accordance with an aspect of the present invention; and

FIG. 9 is a diagram showing the differences between a non-anatomic tissue wedge resection in a lung and an anatomic lobe resection in a lung, in accordance with an aspect of the present invention.

### DETAILED DESCRIPTION OF THE INVENTION

Generally stated, disclosed herein is a surgical instrument for use in resecting diseased tissue from the body of a living being. More specifically, the surgical instrument will typically be used to resect tumors from certain organs within a human or animal body. The surgical instrument generally includes a handle with a trigger grip, an actuation mechanism, two cutting elements, and a guide member.

As used herein, the terms “surgical instrument” or “instrument” may be used interchangeably as they essentially describe the same type of operative instrument. Further, the terms “pin” or “needles” may be used interchangeably to describe the same type of surgical tool. Also described herein is a surgical method for using the surgical instrument with an image guidance system, and a tissue resection kit that is used to provide a plurality of various sized surgical instruments and elongated surgical pins to the operating surgeon that allows them to address the numerous clinical circumstances that may be presented.

In this detailed description and the following claims, the words proximal, distal, anterior, posterior, medial, lateral, superior and inferior are defined by their standard usage for indicating a particular part of a bone, organ or surgical instrument according to the relative disposition of the surgical instrument or directional terms of reference. For example, “proximal” means the portion of an instrument positioned nearest the torso, while “distal” indicates the part of the instrument farthest from the torso. As for directional terms, “anterior” is a direction towards the front side of the body, “posterior” means a direction towards the back side of the body, “medial” means towards the midline of the body, “lateral” is a direction towards the sides or away from the midline of the body, “superior” means a direction above and “inferior” means a direction below another object or structure.

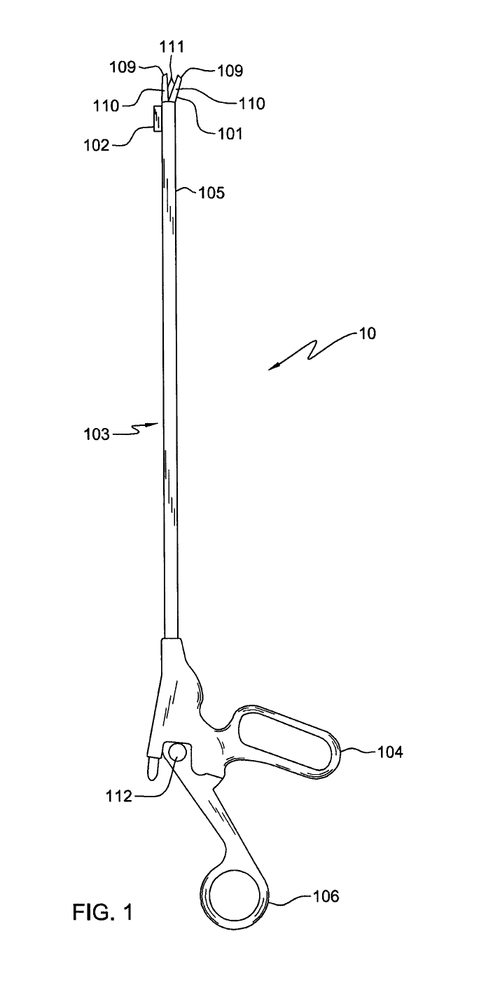

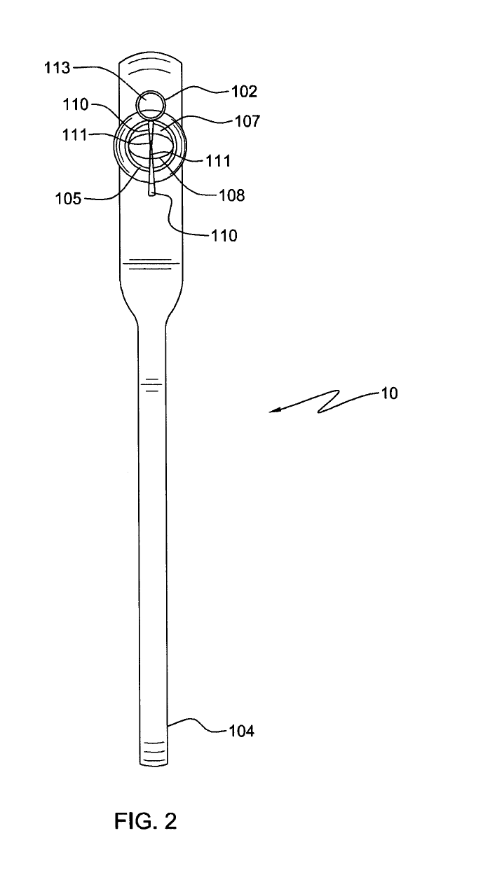

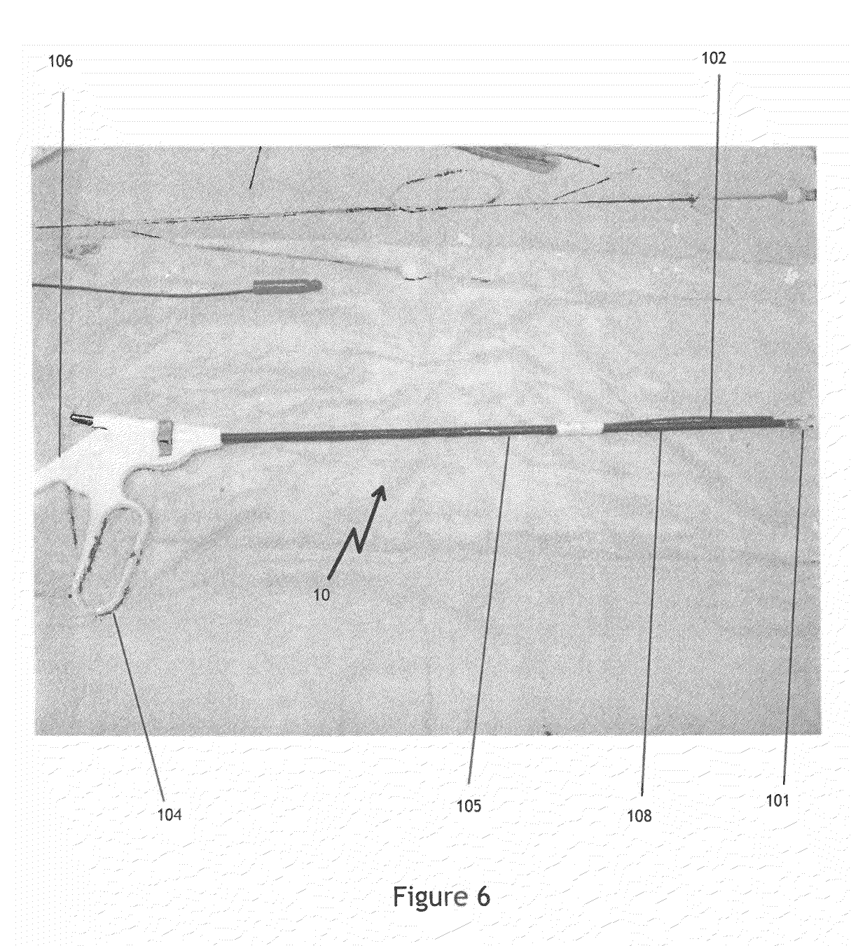

As depicted in FIGS. 1 and 6, the general arrangement of the surgical instrument 10 in accordance with an aspect of the present invention, includes a handle 104 connected to the distal end of an elongate member 105 that extends in a proximal direction from handle 104. Handle 104 may also include a trigger grip 106 that when depressed or released moves the cutting elements 101 that are positioned at the proximal end of elongate member 105. Passing thorough the internal cavity 107 (shown in FIG. 2) of elongate member 105 are the connecting members 108 of the actuation mechanism 103 that are attached to the distal aspect of cutting elements 101 to facilitate movement of cutting elements 101 when trigger grip 106 is depressed. Handle 104 in combination with trigger grip 106 and connecting members 108 may be referred to herein collectively as actuation mechanism 103. Actuation mechanism 103 functions to move at least one, or both cutting elements 101.

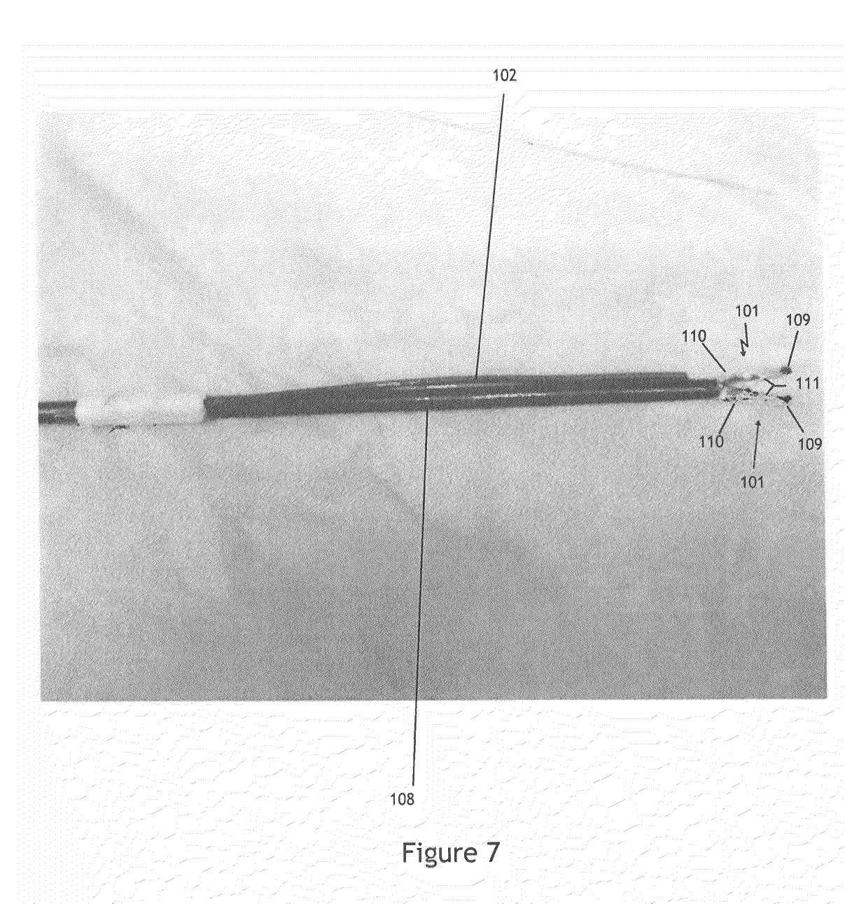

FIGS. 1 and 7 show cutting elements 101 of surgical instrument 10. Cutting elements 101 each generally include a cutting blade 111 that may be straight, serrated, or curved to facilitate resection of tissue. The tips 109 of cutting elements 101 may be configured to facilitate movement of instrument 10 through the tissue when passing along the guide needle 201. Tips 109 may be for example, ridged, pointed, tapered or blunted depending on the particular clinical situation. The configurations of tips 109 are also to facilitate movement of cutting elements 101 relative to the tissue. Cutting elements 101 construct may further include a blade body 110. Body 110 may be configured to be tapered or straight to facilitate passage through and within the tissue. Body 110 shape or configuration that is used in vivo may be determined by the surgeon user and may be determined by the type of tissue and clinical circumstances.

Also shown in FIGS. 1, 6 and 7 is guide member 102, which for example purposes, is disposed along the superior aspect of elongate member 105. Guide member 102 is sized and shaped to accommodate needle 201 or a like elongated rigid structure to facilitate the directing of cutting elements 101 to a targeted location within a body. For example purposes, guide member 102 is shown in FIG. 2 as having a hollow, channel-like structure 113, but it is contemplated that other structural configurations may be used, like rails, tubes, slots, etc. Further, guide member 102 may be integrally attached to an aspect of elongate member 105 or detachable connected (i.e., modular) to allow for movement or repositioning of guide member 102. Guide member 102 may also be internal to elongate member 105, in that for one of the alternative embodiments of surgical instrument 10, a guide needle 201 may pass through internal cavity 107 or channel of elongate member 105 to direct cutting elements 101 to the target location. Further, guide member 102 may be located on the top portion or the side of elongate member 105 or moved more proximate to the proximal or distal aspects of surgical instrument 10. The ability to change the positioning of guide member 102 will allow the surgeon user to accommodate certain anatomic structures and/or clinical circumstances while still optimizing the targeting of cutting elements 101. Guide member 102 may also be placed on the superior, lateral, or medial aspects of body 110 or tip 109 of one of cutting elements 101 to accommodate various clinical situations. Yet further, for an alternative embodiment of surgical instrument 10, guide member 102 may be positioned between or intermediate cutting elements 101, thus, allowing guide needle/pin 201 to pass intermediate two cutting elements 101. It is further contemplated that an alternative embodiment of surgical instrument 10 may include guide member 102 being placed at some location on one of connecting members 108 or between two connecting members 108 of actuation mechanism 103. As described above, guide member 102 may be integrally fixed or detachably connected (i.e., modular) to either of cutting elements 101 or connecting members 108. For these alternative embodiments, the construct of surgical instrument 10 may resemble in some aspects the blades of a pair scissors or a Kelly-clamp like structure with guide member 102 being attached to one of the cutting elements bodies 110, along the shaft portion of a rigid handle that extends from the finger loops to the blade 111 or between the jaws of the connected cutting elements 101.

Although not shown, guide member 102 may also include in its construct a stop mechanism that restricts the length of translation or movement that is possible when guide needle 201 passes through guide member 102 following insertion into the body. The stop mechanism will assist the surgeon user by limiting how far guide member 102 can slide along guide needle 201 and thus, the amount of tissue that cutting elements 101 can resect. For example purposes, the stop mechanism may be the length of cutting elements 101 so that cutting elements 101 may only be moved as far as the tip of guide needle 201. Alternatively, a stop mechanism may be placed along the shaft of guide pin 201 so that the surgeon user could insert guide needle 201 into tissue to a certain depth and then restrict the amount of translation of guide member 102, thereby also restricting the amount of resection by cutting elements 101.

Elongate member 105 shown in FIGS. 1, 6 and 7 may be fabricated from a flexible material to allow for connecting members 108 of surgical instrument 10 to pass through a central internal cavity 107 (see FIG. 2) and be protected from any adjacent internal anatomic structures. Generally, elongate member 105 may be used if surgical instrument 10 is used in conjunction with an endoscope. For certain clinical circumstances, elongate member 105 may be fabricated from a rigid material to support connecting members 108 and provide stability when positioning cutting elements 101. As discussed above, alternative embodiments of surgical instrument 10 may also include various lengths and configurations of connecting member 108 of actuation mechanism 103. Correspondingly, to accommodate the various lengths and sizes of connecting member 108, the length of the protective elongate member 105 will vary depending on the clinical situation and whether surgical instrument 10 is used in conjunction with a minimally invasive apparatus.

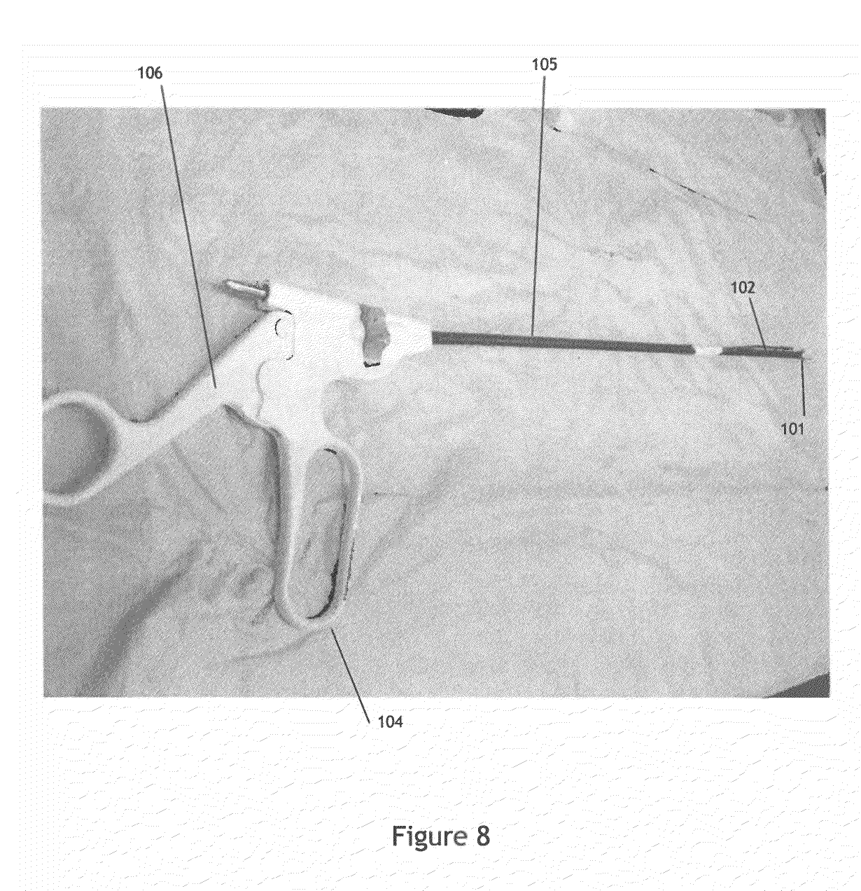

With reference to FIGS. 1 and 8, actuation mechanism 103 typically includes at the distal aspect of surgical instrument 10, handle 104 with trigger grip 106. As shown in FIG. 1, trigger grip 106 will rotate around hinge pin 112 when pressured by the surgeon user's fingers. When depressed, trigger grip 106 causes at least one of cutting elements 101 to move resulting in the resection of adjacent tissue. Alternatively, depression of trigger grip 106 may also cause both cutting elements 101 to move together to resect tissue intermediate these structures. Further, for an alternative surgical instrument where cutting elements 101 have been replaced with a stapling head or a cutting/sealing assembly, depression of trigger grip 106 will cause the stapler head to push out staples into an anvil to seal a gap in adjacent tissue or activate a sealing assembly that functions to close a tissue opening by discharging some sort of sealing material. Although not shown, actuation mechanism 103 may also be configured as two moveable handles or loops that are similar in configuration to scissor handles, or alternatively, as two parallel extensions or handles that when squeezed results in the movement of one or both of cutting elements 101. In the event surgical instrument 10 is used in conjunction with an endoscope or other minimally invasive apparatus, it would understood by one skilled in the art that trigger grip 106 or handle 104 of actuation mechanism 103 is positioned outside of that scope or apparatus enabling the surgeon user full access to manipulating the various configurations of the handles and/or trigger grips.

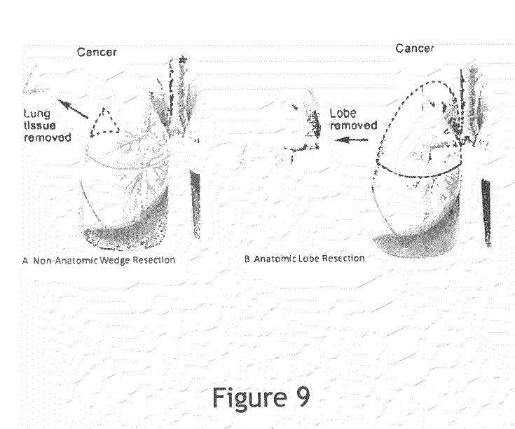

As shown in FIG. 9, one of the purposes of surgical instrument 10 and the corresponding inventive surgical method 400 is to remove a wedge of diseased tissue from the target organ or body part rather than removing adjacent healthy tissue in addition to the targeted diseased tissue.

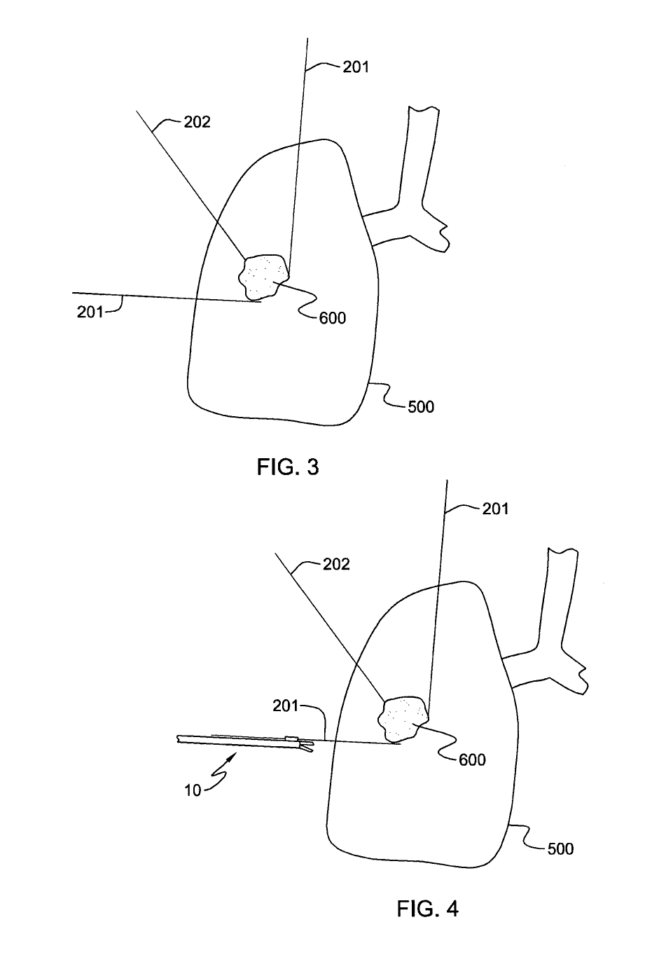

FIGS. 3 and 4 depict the placement of guide pins 201 along the margins of the diseased tissue or tumor 600 to establish cutting planes in, for example, a lung 500. Diseased tissue or tumor 600 is held under tension or in traction by pin 202. As seen in FIG. 4, following placement of pins 201 and 202, the surgeon user can then guide surgical instrument 10 along both guide pins 201 to resect the tissue around tumor 600 and allow for the extraction of the diseased tissue from the body.

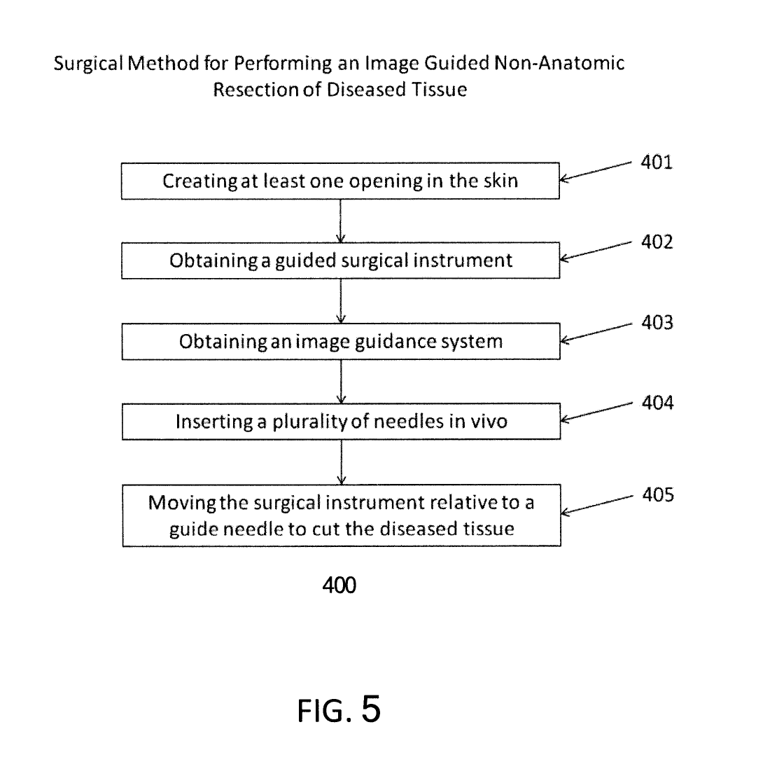

The diagram shown in FIG. 5 describes the suggested steps of the surgical method 400 for using surgical instrument 10 while performing an image-guided, non-anatomic resection of diseased tissue. The initial step 401 may include surgically creating at least one opening in the skin of a patient with the at least one opening being preferably proximate to the location of the diseased tissue. Alternatively, the surgeon user may create a skin nick to percutaneously insert guide needles 201 and traction pin 202 or enable the surgeon user to insert a trocar (not shown) that is then used to insert needles 201 in close proximity to targeted tissue 600.

The method may include the step 402 of obtaining a guided surgical instrument 10 that has two cutting elements 101, guide member 102 and actuation mechanism 103. An alternative embodiment of surgical instrument 10 may be used for the method with cutting elements 101 being replaced by a stapling head or a sealing/cutting assembly. The structural elements of such a surgical instrument would be the same other than cutting elements 101 being exchanged for a stapling head or sealing mechanism. These components have been more fully described above and for brevity sake will not be described again here.

Surgical method 400 also includes the step of obtaining an image guidance system 403. Image guidance systems that are well known in the art include for example, x-ray, fluoroscopy, ultrasound, CT or MRI. The image guidance system will be used to assist the surgeon user with the placement of the plurality of needles and surgical instrument.

As shown in FIG. 5, the method 400 further includes the step of the surgeon user inserting a plurality of needles through the skin and into the targeted tissue region 404. FIG. 3 depict a first needle 202 following insertion directly into the diseased tissue to hold and capture the targeted tissue, which for example purposes has been described as a tumor 600. The image guidance system is used to correctly place traction needle 202 that may have a proximal end that is configured as a claw, grip, hook or other similar design that functions to grab onto target tissue 600. Glue or other adhesive material may also be dispensed at the proximal end of traction needle 202 to attach to target tissue 600.

Again, using the image guidance system, a second needle 201 and a third needle 201 may also be inserted along the margins of diseased tissue 600, and thereby establishing the resection boundaries for the wedge resection procedure. It should be noted that additional boundary guide needles 201 and traction needles 202 may be used depending on the clinical circumstances that are presented. For clinical situations, like for example, cases where tissue is being removed from a lung, an additional needle, like a pleural needle may also be inserted to inject air into the body cavity to deflate the lung while placing tension on traction needle 202.

Surgical method 400 may also include the step 405 of moving guide member 102 along the shaft of either the second or third (guide) needle 201, thereby allowing the surgeon user to be able to actuate at least one of cutting elements 101 of surgical instrument 10 and cut the encountered diseased tissue 600 as shown in FIG. 4. Guide needles 201 define a plurality of cutting paths that are positioned along the margins of the diseased tissue to allow cutting elements 101 be directed to cut the diseased tissue in the preferred wedge shape (see FIG. 9). Guide needles 201 are generally rigid in structure with a constant diameter so as to permit guide member 102 of surgical instrument 10 to slide in a precise and directed fashion along the cutting or resection trajectory as established by the axis of corresponding guide needles 201.

Although not shown it is contemplated that an alternative embodiment of the surgical instrument may be used following the resection of diseased tissue or in the case where lacerated tissue requires repair. This alternative surgical instrument would be modified in a manner where cutting elements 101 have been replaced by a stapler head or cutting/sealing assembly at the proximal end of the surgical instrument, the surgeon user will move the guide member 102 along one of the guide needles 201 until the surgical instrument is in position that when actuation mechanism 103 is actuated, the proximate tissue is stapled or a gap in the adjacent tissue is sealed closed.

Although not shown, it is contemplated that an additional step in surgical method 400 may include the insertion of an internal imaging device (e.g., camera) that allows the surgeon user to visualize and observe the cutting elements and/or stapler head in position prior to actuation of the actuation mechanism.

It should be understood by those skilled in the art that surgical method 400 and the use of surgical instrument 10 described herein may be performed using either anterior, posterior or lateral approaches to the surgical site. Also, the surgical instrument 10 may be used in a minimally invasive surgical approach, percutaneous approach or in a modified open approach.

Another aspect of the invention is a tissue resection kit that includes multiple configured surgical instruments 10 with each having various sizes, lengths and stiffnesses of connecting members 108, guide members 102, elongate members 105 and cutting elements 101. It is understood that surgical instrument 10 may be modular in design, thereby allowing for the surgeon user to attach and detach connecting members 108, guide member 102 and cutting elements 101. Whether the surgical instrument is modular or non-modular, the kit may also include various types of cutting elements 101 with different configured blades 111, (e.g., curved, straight, serrated), bodies 110, (e.g., tapered, straight) or tips 109, (e.g., blunt, pointed, tapered). In addition, the kit may contain various sized stapler heads and cutting/sealing assemblies.

Further, the kit may include a plurality of lengths, diameters and stiffnesses of traction and guiding needles 201, 202. The fraction or tensioning needles 202 may have a variety of configured proximal ends for holding onto the tissue. These ends may include claws, hooks, rakes or adhesive heads. Having the kit available will allow the surgeon user to pick and choose the appropriate sized surgical instrument 10 and corresponding traction and guide needles 201, 202 that best fits the presented clinical situation or to address a certain anatomical deformity found in a patient. All embodiments of the surgical instruments 10 in the kit will typically include the above described construct elements that for brevity sake, will not be discussed again here and include the same structural and functionality characteristics as described previously herein.

Although the various embodiments have been depicted and described in detail herein, it will be apparent to those skilled in the relevant art that additional modifications, and substitutions can be made without departing from its essence and therefore these are to be considered to be within the scope of the following claims.

What is claimed is:

1. A surgical instrument for resecting tissue, the surgical instrument comprising:
a first cutting element and a second cutting element;
a guide member; and
an actuation mechanism;
wherein the guide member is coupled to at least one of the first cutting element, the second cutting element and the actuation mechanism with the first cutting element and the second cutting element being operatively connected to the actuation mechanism, the actuation mechanism adapted to actuate at least one of the first cutting element and the second cutting element to facilitate the resection of tissue.

2. The surgical instrument of claim 1, wherein the actuation mechanism comprises a handle, trigger grip and at least one connecting member adapted to couple the trigger grip to at least one of the first cutting element and the second cutting element to facilitate movement of at least one of the first cutting element and the second cutting element when the trigger grip is actuated.

3. The surgical instrument of claim 1, further comprising an elongate member, wherein the elongate member has a proximal end and a distal end adapted to connect to the handle.

4. The surgical instrument of claim 3, wherein the elongate member is hollow and configured to receive at least one connecting member.

5. The surgical instrument of claim 3, wherein the elongate member is fabricated from at least one of a flexible material and a rigid material.

6. The surgical instrument of claim 1, wherein the first cutting member and the second cutting member comprise a blade, a body and a tip.

7. The surgical instrument of claim 6, wherein the blade comprises at least one of a serrated edge, a straight edge and a curved edge.

8. The surgical instrument of claim 6, wherein the tip comprises at least one of a ridged shape, a blunt shape, a tapered shape and a conical shape.

9. The surgical instrument of claim 6, wherein the body is at least one of straight and tapered.

10. The surgical instrument of claim 3, wherein the guide member is attached substantially parallel to the elongate member and positioned at the at least one of the distal end, the proximal end and intermediate the distal end and proximal end of the elongate member.

11. The surgical instrument of claim 2, further comprising a plurality of connecting members, wherein the guide member is positioned between and substantially parallel to the plurality of connecting members.

12. The surgical instrument of claim 1, wherein the guide member further comprises a stop to restrict movement of the surgical instrument.

13. A surgical method for performing an image-guided, non-anatomic resection of diseased tissue, the method comprising:
creating at least one opening in the skin of a patient;
obtaining a guided surgical instrument comprising:
a first cutting element and a second cutting element;
a guide member; and
an actuation mechanism;
wherein the guide member is coupled to at least one of the first cutting element, the second cutting element and the actuation mechanism with the first cutting element and the second cutting element being operatively connected to the actuation mechanism, the actuation mechanism adapted to actuate at least one of the first cutting element and the second cutting element to facilitate the resection of tissue.
obtaining an image guidance system;
inserting a plurality of needles into the patient through the at least one opening using the image guidance system to direct placement of the plurality of needles, the plurality of needles comprising;
a first needle being inserted into the diseased tissue of the patient; and
a second needle and a third needle being inserted into the patient, the second and third needle being configured to receive the guide member of the surgical instrument and are oriented in a manner to define a cutting axis to be followed by the first and second cutting elements of the surgical instrument; and
moving the guide member relative to the second or third needle, thereby allowing the operator to actuate at least one of the first and second cutting elements of the surgical instrument.

14. The method of claim 13, further comprising cutting the diseased tissue with at least one of the first and second cutting elements.

15. The method of claim 13, wherein inserting a plurality of needles further comprises inserting a fourth needle in the pleural space to deflate a lung.

16. The method of claim 14, further comprising inserting an imaging device within a body cavity to visualize the cutting of the diseased tissue.

17. The method of claim 13, wherein the first needle is attached to the diseased tissue by at least one of an adhesive material, a claw and a hook.

18. A tissue resection kit, the kit comprising:
a plurality of surgical instruments for use in resecting targeted diseased tissue, each of the plurality of surgical instruments comprising:
a first cutting element and a second cutting element;
a guide member; and
an actuation mechanism;
wherein the guide member is coupled to at least one of the first cutting element, the second cutting element and the actuation mechanism with the first cutting element and the second cutting element being operatively connected to the actuation mechanism, the actuation mechanism adapted to actuate at least one of the first cutting element and the second cutting element to facilitate the resection of tissue; and
a plurality of elongate needles.

19. The tissue resection kit of claim 18, wherein the plurality of elongate needles have different sized diameters, overall lengths and proximal end configurations.

20. The tissue resection kit of claim 18, wherein the kit further comprises an image guidance system.
